(19) United States
(12) Patent Application Publication
(10) Pub. No.: US 2017/0368183 A1
(43) Pub. Date: Dec. 28, 2017

(54) TISSUE REPAIR SYSTEM

(71) Applicant: DSM IP ASSETS B.V., TE HEERLEN (NL)

(72) Inventors: Gino BRADICA, Mullica Hill, NJ (US); Ryan Andrew SASKA, Exton, PA (US); Christopher DIJIACOMO, Exton, PA (US); Timothy A. RINGEISEN, Exton, PA (US)

(21) Appl. No.: 15/678,337

(22) Filed: Aug. 16, 2017

Related U.S. Application Data
(63) Continuation of application No. 13/732,180, filed on Dec. 31, 2012, now abandoned.

(51) Int. Cl. A61K 47/42; A61K 35/16; A61L 27/60; A61L 27/58; A61K 35/19; A61L 27/36; A61L 27/24; A61K 9/00; A61K 35/28; A61L 26/00; A61L 27/48

(52) U.S. Cl. CPC A61K 47/42; A61L 26/0095; A61K 35/16; A61L 27/60; A61L 27/58; A61L 27/48; A61L 27/3616; A61L 27/24; A61K 9/0024; A61L 26/009; A61L 26/0057; A61L 26/0033; A61K 35/28; A61K 35/19; A61L 2400/04; A61L 2400/06

(57) ABSTRACT

An implant for promoting accelerated wound healing. The implant comprises a non-flocculating fiber material, admixed with a settable fluid. The fiber component typically will have short fiber lengths, so as to avoid forming entangled masses or clumps when mixed with a fluid. In an embodiment, the fiber material is native collagen fibers and the settable fluid is an isolated blood fraction, such as platelet rich plasma and platelet poor plasma. The native collagen fiber retaining the native crosslinks of the source tissue and providing an architectural and structural scaffolding for advancing cellular infiltration. The wound healing implant will accelerate the bodies healing process, to provide better healing and less scar tissue of the wound site.

### BACKGROUND OF THE INVENTION

### 1. Field of the Invention

The present invention pertains to methods and materials for hemostat and tissue regeneration. More particularly, it pertains to methods and materials for forming high strength blood clots at the site of tissue lesions, the strength enabling the suturing of the clot to the tissue to be repaired.

### 2. Discussion of Related Art

As generally known, platelet rich plasma (PRP) is an autologous material, separated from whole blood, such as through the technique of density gradient centrifugation, for example, by using the devices and techniques described in U.S. Pat. No. 8,317,672. The preparation of PRP results in increased concentrations of the platelets and growth factors (GF) found in whole blood.

While it is known to provide PRP to a wound site, the application of PRP in a liquid state to a wound site often presents difficulties, as liquid PRP, when injected or otherwise delivered to a wound site, will “washout”, as body fluids or irrigating fluids at the wound site dilute or carry away the liquid PRP, prior to the healing cascade being completed. Thus, a technique had been previously developed to provide gelled PRP to a wound site, such that the PRP has been activated and is in the form of a gelled clot, with hydrogel properties, and thus more resistant to washout away from the wound site. However, the prior art techniques of preparing a gelled PRP mixture have had many shortcomings. For example, prior art gelled PRP is unable to withstand fixation with sutures, to provide a secure attachment of the treatment material to the wound site, as the suture would simply pull through (tear) the gelled PRP. What is needed is a gelled PRP that is able to be sutured in place using traditional techniques, to ensure adequate contact between the wound site and the treatment PRP material. Furthermore, the prior art gelled PRP fails to provide, and the various embodiments described herein do provide, a scaffold material that maintains an inner architecture, characterized by interconnected interfiber space, which will be filled with chemotactic gel created by the fractionated blood component, such that cells will be able to infiltrate into and penetrate throughout the scaffold. Additionally, the prior art gelled PRP does not provide native collagen binding sites within the gelled PRP, as can be found in the embodiments described herein, where such native collagen binding sites are necessary to allow the various growth factors released from the activated PRP to bind to the scaffold material, and serve as an extended release mechanism to prolong the delivery of beneficial healing growth factors, not only to the wound site, but also throughout the entirety of the treatment material. What is needed is a composition that addresses the aforementioned shortcomings of the prior art.

Collagen has been recognized in the prior art as a replacement material for thrombin. Collagen is a structural fibrous protein, and is a main protein component of a mammalian connective tissue. It makes up 30% or more of total proteins. The collagen provides shape, strength and flexibility to the tissue, and has various functions such as tissue scaffolding, cell adhesion, cell migration, blood vessel production, tissue morphogenesis, tissue repair, and the like. Collagen, as a strong agonist of platelets, activates the platelets, and causes platelet aggregation. While reconstituted fibrillar collagen, as described by Park et al. in U.S. Patent Application Publication No. 2012/0201897, more strongly induces platelet aggregation and supports greater platelet adhesion than soluble collagen, it lacks the mechanical integrity provided by native collagen fiber cross-links. Additionally collagen which has been reduced to a lower order form loses the organized internal architectural structure and native binding sites that would be provided by collagen that is in native fibrous form.

However, there has been no composition previously described for inducing tissue regeneration by activating PRP with native collagen fibers, and optionally, a calcium ion, such as can be provided by the addition of a calcium salt (e.g., calcium chloride, or calcium carbonate).

### SUMMARY OF THE INVENTION

The present invention is useful for the healing of tissue wounds, by providing a fibrous scaffold material combined with blood components, especially blood fractions rich in platelets, such that the combination forms a regenerative complex. The blood components form a hydrogel in combination with the fibrous scaffold, which preferably provides native binding sites of collagen fibers, and further provides an architectural and structural scaffolding suitable for cellular infiltration. This promotes wound healing and advances the typical wound healing response, such that the wound will more quickly heal, such that there will be less scar tissue created, and better healing of the wound site.

In an embodiment, the implant is a tissue scaffold comprising a fibrous collagen component, where the collagen is modified in a manner such that it resists flocculation. The non-flocculating collagen is combined with a settable fluid.

In an embodiment, the settable fluid may be fractionated blood components, especially platelet rich plasma. Alternatively, the settable fluid may comprises platelet poor plasma or combinations of platelet rich plasma and platelet poor plasma. In another embodiment, the settable fluid is bone marrow aspirate.

In an embodiment, the fibrous collagen component is native fibrous collagen. The fibrous collagen may be of a fiber length less than 4 mm, may be less than 2 mm, may be less than 0.75 mm, or may be between 0.01 mm and 1 mm.

In an embodiment, the fractionated blood components are prepared by the separation of platelets and/or plasma from whole blood. In an embodiment, the platelets and the plasma are provided in a ratio between 1:2 and 1:10 by volume.

In an embodiment, the platelets in the composition will react with the collagen fibers to form a clot, where the collagen fibers are arranged as a fiber reinforcement within the clot. The fibers may form a matrix within the settable fluid, that is suitable from cell migration, attachment and proliferation.

In an embodiment, the tissue scaffold may further have biologically active agents, such as drugs, cells, growth factors, that are incorporated into the material as an additive. In an embodiment, the tissue scaffold may further have non-fiber particulates added, such as synthetic and natural polymers, ceramics, metals, bioglass, incorporated into the material as an additive.

The tissue scaffold may be implanted prior to the setting of the settable fluid component, or alternatively, may be implanted after setting of the settable fluid component.

In the method of preparing an embodiment that is a tissue scaffold, the method requires providing a blood sample, and native insoluble fibrous collagen. The blood sample is processed in a centrifuge device to fractionate the blood into component fractions, such that at least one of the blood fractions can be collected. The collected blood fraction can then be added to the native insoluble fibrous collagen, whereupon it reacts to form a clot.

In another embodiment, the collagen fibers described herein may be presented as a dry powder, or alternatively in a partially hydrated form, to the wound site, and serves as a hemostat material, wherein blood from the wound site will rapidly absorb into the collagen, and react to form a clot.

### DETAILED DESCRIPTION

The present invention has been made in view of the above-mentioned problems, and provides a composition for inducing tissue regeneration by providing a regenerative complex containing native fibrous collagen and fractionated blood components, and a method of manufacturing the same.

There are three overlapping stages to wound healing: (1) inflammatory, (2) proliferative, and (3) remodeling, as will be discussed below. The present invention has been conceived to emulate certain parameters within a wound healing response, so as to provide a temporary scaffold material along with the autologous platelets, which provide growth factors and cytokines, which, in turn initiate the wound healing at a stage beyond the early stages of wound healing, by advancing the healing to the later stages.

The first stage of wound healing is inflammation, and it is the initial response to tissue injury. The effect of inflammation is to provide rapid hemostasis, and begin the sequence of events that leads to regeneration of the damaged tissue. A hematoma is a collection of blood, typically clotted, that forms to fill the wound space. A hematoma forms by one of two pathways: intrinsic and extrinsic. The intrinsic pathway is initiated by damage or alteration to the blood itself, whereas the extrinsic pathway is initiated by contact of blood with factors that are extraneous to the blood (e.g., damaged tissue). Both pathways involve a cascade reaction sequence. Although both pathways begin differently, they converge and share many of the later steps in the reaction, whereby inactive factors become activated (e.g. growth factors and cytokines are released by activated platelets and other cells, resulting in cell migration, proliferation, differentiation and matrix synthesis) which, in turn, catalyze the formation of other products from precursors that go on to catalyze subsequent reactions, eventually leading to the formation of a formal clot. In order to form a clot, calcium ions are required for the reaction to proceed to completion.

The second stage of wound healing is the proliferative phase, which is largely controlled by cells removing damaged, necrotic tissue, and replacing it with living tissue that is specific to the local tissue environment (e.g., bone, cartilage, fibrous tissue, skin, etc.). The mesenchymal stem cells, which are recruited to the wound area, differentiate into osteoblasts, fibroblasts, chondrocytes, and other cell types as required to generate the appropriate type of tissue. Local factors, including the presence of growth factors and cytokines, hormones, nutrients, pH, oxygen tension, and the electrical and mechanical environment, mediate the appropriate differentiation. Growth factors are needed to initiate the proliferation phase. These growth factors are critical for any wound to heal, and are involved in every phase of wound healing. Growth factors are contained in the alpha granules of platelets, as well as other cells, such as macrophages and endothelial cells. Platelet growth factors are responsible for the early migration of cells to the injury site and the triggering of mitosis of these cells once at the site.

The third stage of wound healing is the remodeling phase, which is characterized by the creation of newly generated tissue, which reshapes and reorganizes to more closely resemble the characteristics of the original tissue. Changes that occur include a reduction in cell density and vascularity, removal of excess repair matrix that has been laid down during the proliferative phase, and orientation of the collagen fibers of the repair matrix along lines of stress to maximize strength. This final stage of healing typically require years for completion.

The body has a limited capacity, depending on the extent of the injury, to assist in the healing of a wound site. The applicants believe the prior art methods of treating wound sites fall short of emulating these healing stages by not providing the needed structure to the temporary matrix to maintain a regenerative space and provide a scaffold for cell migration and proliferation. The structure provided by the present invention, maintains the native collagen binding sites in the structure and is believed to help bind growth factors, thus establishing chemotactic gradients for cell recruitment, as well as a storage pool of growth factors that can be secondarily released by the action of the naturally present metalloproteases on the structure. Furthermore, the structure according to the present invention provides adequate regenerative space for cellular regenerative events. In contrast to the normal clotting sequence, where the platelets form a clot as a dense aggregate, that quickly becomes impenetrable to cellular infiltration.

Platelet-derived factors (growth factors include platelet derived growth factor (PDGF-AB), transforming growth factor-b1 (TGF-b1), vascular endothelial growth factor (VEGF), epidermal growth factor (EGF), insulin-like growth factor (IGF) and the like) can influence cellular growth, morphogenesis and differentiation. Due to the establishment and maintenance of the regenerative space by the structure of the present invention, the ability of platelets to release within this space makes the structure a therapeutic source of growth factors and cytokines that can increase the production and remodeling of collagen thereby accelerating the natural healing processes.

The structure of one embodiment of the present invention will be made of native fibrous collagen, which is made up of bundles of collagen fibrils, which in turn are semicrystalline aggregates of collagen molecules. These native fibers are preferred for their architectural and mechanical characteristics, as they will provide enhanced physical properties when compared to lower orders of collagen. The native fibrous collagen is used as a structural element, wherein a source of collagen tissue, typically an allograft or xenograft source (e.g., porcine, bovine, caprine, piscine, ovine, etc.) has been prepared by chemically cleansing the tissue of non-collagenous substances while still maintaining the architectural structure of the collagen and then mechanically disrupting the resultant material, thus breaking it down into fibers which still maintain the unique architecture of the specific tissue from which it was sourced (i.e. hide, tendon, intestine, etc). Chemical reduction of the collagen to lower order forms (e.g., fibrillar, soluble) should be avoided, as it destroys the nativity of the collagen. The fibers are then further prepared in a manner such that the fibers will not flocculate by forming a composition characterized by lumps or masses, but rather it is preferable to create a uniform (non-flocculated) distribution of fibrous materials, when mixed with other solid or liquid materials. To avoid flocculation of the native collagen fibers, the fibers are of a short length, typically less than 4 mm, typically less than 2 mm, typically less than 0.75 mm, and typically between 10 microns and 1 mm. Importantly, the collagen fibers of the present invention are treated in a manner that preserves the native cross-links that are found in native collagen, and avoids disrupting the collagen structure, so as to avoid reducing the organizational level of the collagen to a fibrillar, soluble, or tropocollagen level. Additional aids in avoiding entangled lumps or masses of fibers is by using agents that stiffen the fibers, wherein after bending or flexure, the fibers will return back to their original state. It is recognized that in the practice of the present invention, one may beneficially combine the fibers described above with other collagen forms, such as fibrillar or soluble collagen forms, however, it is important that there remain native fibrous collagen, for the reasons described herein. The native fibrous collagen as used in the present invention is not to be confused with, reconstituted soluble or other lower forms of collagen, such as tropocollagen. These lower and reconstituted forms of collagen lose physical integrity and the natural binding sites during the unraveling of the collagen triple helix, which in turn leaves the base molecule with exposed telopeptides, which are thought to be responsible for inflammatory responses.

In the practice of the present invention, the native collagen fibers will be combined with a settable fluid; preferably the native collagen fibers will be prepared with platelet rich plasma (PRP) that has been separated from a small amount of whole blood, for example, by density gradient centrifugation. Alternatively, the settable fluid may be whole blood, bone marrow aspirate or lipoaspirate or derivatives thereof. Additionally, the native collagen fibers can be partially hydrated with other non-settable fluids prior to exposure to the settable fluid. This additional fluid can be added for multiple reasons obvious to one skilled in the art including, but not limited to: reducing the fiber packing density of the final set construct; incorporation of biologically active agents (drugs, cells, growth factors, etc) or particulates (ceramic, metal, glass, polymer, etc) which are dissolved into or suspended within the additional fluid; to carry other substances that accelerate or decelerate the setting time of the settable fluid; to modify handling properties by reducing static effects on the dry native collagen fibers or to facilitate injection through a delivery tube, syringe or needle.

In an alternative form, the native collagen fibers as described herein make a unique hemostatic agent which can be, in a dry state, placed into or onto a bleeding wound. Alternatively the native collagen fibers can be partially hydrated and packed or injected into, or onto, a bleeding wound wherein the unique fibers function as a hemostat.

It is also obvious to one skilled in the art that biologically active agents and/or particulates can also be incorporated into either or both of the dry fibers or settable fluid. Examples of biologically active agents can be found in table 2 and examples of particulates can be found in table 3.

In accordance with an aspect of the present invention, there is provided a composition, and the method of manufacture thereof, for inducing tissue regeneration by providing a regenerative complex containing fibrous collagen and fractionated blood components. The method including the steps of: separating PRP from whole blood; optionally treating the separated PRP with an anticoagulant, which will extend the time the blood may be stored prior to separation or use by preventing premature clotting. Where an anticoagulant is used, it may be beneficial to negate the effects of the anticoagulant prior to admixing with collagen, such as may be achieved by mixing the anticoagulated PRP with a calcium ion. The calcium ion may be provided by addition of a calcium salt, or addition of solution containing calcium ions; typically calcium ion is provided by a calcium chloride solution, though other sources of calcium may be used, such as calcium carbonate. The separated PRP may then be mixed with native fibrous collagen.

The PRP is admixed initially as a liquid, and due to the clotting cascade, will set within minutes to a hydrogel, when contacted with the collagen fibers. The liquid PRP is added to the collagen fibers, and upon implanting into a wound site of a living being, the PRP is useful for signaling the early migration of cells to the injury site and the triggering the mitosis of these cells once at the site. The PRP can be injected as a mixture with native fibrous collagen. In other words, PRP as an autologous material and native fibrous collagen are gelled to create a reinforced matrix bound to a scaffold of fibrous collagen extending throughout the entirety of the regenerative complex. The collagen scaffold provides for an open porous architecture that allows cellular infiltration through the gel of the reinforced matrix, and the preserved nativity of the collagen ensures that the growth factors will readily bind to the collagen, thus serving to provide for an extended release of the growth factors from the degranulated alpha granules of the platelets, and further, thus allowing for enhanced cellular recruitment into the scaffold. . It is believed that the healing would occur more quickly, and results in better healing, with less scarring, than in wound healing without using native fibers mixed with PRP.

The PRP may be conveniently and quickly separated on site for a clinical procedure, and is injected or placed into the wound site in a mixture, optionally with a calcium ion (e.g. calcium chloride solution, calcium carbonate, etc.), with native fibrous collagen, so that effective tissue regeneration can be achieved for severely injured patients or patients undergoing repetitive operations.

In an alternative embodiment, the PRP collected may be applied to a region requiring tissue-regeneration as a mixture with native fibrous collagen that has been further enhanced with additional cross-links, and optionally calcium ion (e.g., calcium chloride solution, calcium carbonate, etc.). The further cross-linked native fibrous collagen similarly activates the platelets within the PRP, inducing growth factors useful for tissue regeneration from the PRP gel. This is effective to conveniently and quickly achieve tissue regeneration. It is believed that providing the additional cross-links results in a material that is further mechanically enhanced to withstand clinical procedures, for example enhanced resistance to washout or suture pullout. The further cross-linking may be performed by methods known in the art, including exposure to various cross-linking agents (e.g., dopaquinone, embelin, potassium embelate and 5-O-methyl embelin, gluteraldyhyde, 1-ethyl-3-(3-dimethyl aminopropyl) carbodiimide hydrochloride, dehydrothermal crosslinking, etc.)

Cross-linking of fibrous collagen in a fully hydrated state, in an excess of fluid, wherein linkages generally do not form between individual fibers, forms a stiff, but bendable fiber with shape memory, wherein after bending or flexure, the fibers will seek to return back to their original state, when the bent fiber is in a hydrated state. Although not fully understood, it is believed that the collagen fibers exposed to cross-linking agents, while in an excess of fluid, are believed to form intra-fiber bonds that accommodate the fluid held within the fiber prior to the cross-linking Thus when dehydrated, and then subsequently exposed to fluids, the fibers do not just wet on their surface, but rather imbibe the fluid, and the fibers then return to their original hydrated conformation. In doing so, the fibers concentrate any non-fluid components (i.e., platelets, cells, etc) on the surface of the fiber. Additionally, it is believe that the water imbibed by the fiber allows the fiber to be both stiff and retain the attribute of shape memory if bent. These unique characteristic provided to the fibrous collagen, which have native cross-links, by exposing the hydrated collagen fibers to a cross-linking solutions, while generally avoiding any cross-linking between fibers, produces a novel material for both use as a hemostatic material and in formation of a regenerative complex when exposed to a settable fluid component.

The reinforced matrix provided with native collagen fibers, as an agonist with PRP, can release a similar or larger amount of growth factors than can be achieved through the typical healing pathway of the body. This induces effective tissue regeneration.

Furthermore, the PRP blood component when injected in a mixture, optionally with a calcium ion (e.g., calcium chloride solution, or calcium carbonate, etc.), with native fibrous collagen into all regions requiring defect treatment or wound healing, will thereby provide for effective tissue regeneration. Optionally, in external defects or wounds, where the regenerative complex may be prone to desiccation, a covering may be applied to mitigate such events. This covering may be performed by methods know in the art, including the application of silicone, hydrogels, non-adherent dressings, extracellular matrixes (ECM), etc. to form a barrier over the regenerative complex.

The inventive compositions described herein may be beneficially applied in many clinical fields. For example, in cardiology, where the composition may be directly injected into cardiac muscle so as to improve cardiac function, or applied to wound closure sites in order to reduce the potential for deep chest infections. In dentistry, the composition may be applied to scaffolds for tissue and bone repair, such as third molar extractions. In EN&T fields, the composition may be useful for assisting the healing of, for example, cleft pallet, sinus augmentation, etc. For cranial maxillofacial applications, the composition may be applied during reconstruction of mandibular and maxillary bone structures. In the field of neurosurgery, the composition may be applied to nerve endings in order to initiate a healing response. In the field of ophthalmology, the composition may be useful to help heal tissue, such as corneal tissue or alternatively the composition may be applied for ocular surface syndrome, subsequent to LASIK surgery. In orthopedic reconstruction, the composition may be used to coat the surgical site to minimize potential infection and to reduce blood loss. When used in plastic and reconstructive surgery, the composition may be used to accelerate healing, for example, in hair restoration procedures, skin flap healing, and skin wrinkle reduction. In the field of sports medicine, the composition may be used to help the healing in soft tissues. In the field of obstetrics and gynecology, the composition may be used to minimize potential for surgical site infections and postoperative dehiscence. For general wound care, the composition may be applied directly to a wound site, such as pressure ulcers, chronic wounds, lacerations, etc., in order to initiate a healing response (cellular infiltration and angiogenesis).

### EXAMPLE 1

An embodiment of the current invention includes using the fibrous collagen described in Evans et al. U.S. Pat. No. 7,166,133 and mechanically milling the fiber material to a particle size of less than 2 mm., the particularized native fibrous collagen is then placed in a container of known size and shape.

A ratio of platelet rich plasma (PRP) and platelet poor plasma (PPP) is prepared, by separation of blood components from whole blood, such as through the technique of density gradient centrifugation, for example, by using the devices and techniques described in U.S. Pat. No. 8,317,672. The fractioned blood components preferably may comprise platelets (PRP) and plasma (PPP) between 1 to 2 and 1 to 10 volume ratio.

The native fibrous collagen material and fractioned blood components may be combined at weight ratios ranging from 1:100, to 5:3, and may be mildly agitated to confirm even distribution of the materials. This mixture may be placed into a container which will impart a desired shape to the resultant regenerative complex.

The resultant product is a regenerative complex with the properties described herein.

Many materials can be used to supplement the regenerative complex, or a portion thereof, of the embodiments of Applicants' invention. Biocompatible polymers (e.g., collagen, chitosan, alginate, polylactide-co-glycolide, polyurethane, polyethylene) are preferred for use in this invention. As previously described, collagen, and most specifically native fibrous collagen, is a preferred structural constituent of the regenerative complex. Additionally, biocompatible resorbable synthetic polymers may be incorporated into the regenerative complex, such as, but not limited to, those listed in Table 1. However, virtually any biodegradable and/or biocompatible material may be used with the present invention.

In general, the implant of this invention may further be enhanced by the addition of one or more biocompatible materials (e.g. polymer, metal, ceramic) that will act to treat the wound and further add to the fiber scaffold of the regenerative complex, for the in-growth of tissue. The implant may contain a depot of material (e.g. calcium salts, collagens, cytokines, drugs, etc.) for assisting the in-growth of cells and act as a carrier for other constituents (e.g., see tables 2 and 3). Some embodiments of the invention also incorporate cells or other biological constituents for providing the basic building blocks for tissue regeneration.

The device of the subject invention (e.g. implant, delivery system) may contain or deliver one or more biologically active or pharmaceutical agents (i.e., therapies), such as but not limited to those disclosed in Table 2.

TABLE 1
Examples of Additional Biodegradable
Polymers for Use in Construction of the Fiber Matrix of this Invention
Aliphatic polyesters
Cellulose
Chitin
Collagen
Copolymers of glycolide
Copolymers of lactide
Elastin
Fibrin
Glycolide/l-lactide copolymers (PGA/PLLA)
Glycolide/trimethylene carbonate copolymers (PGA/TMC)
Hydrogel
Lactide/tetramethylglycolide copolymers
Lactide/trimethylene carbonate copolymers
Lactide/ε-caprolactone copolymers
Lactide/σ-valerolactone copolymers
L-lactide/dl-lactide copolymers
Methyl methacrylate-N-vinyl pyrrolidone copolymers
Modified proteins
Nylon-2
PHBA/γ-hydroxyvalerate copolymers (PHBA/HVA)
PLA/polyethylene oxide copolymers
PLA-polyethylene oxide (PELA)
Poly (amino acids)
Poly (trimethylene carbonates)
Poly hydroxyalkanoate polymers (PHA)
Poly(alklyene oxalates)
Poly(butylene diglycolate)
Poly(hydroxy butyrate) (PHB)
Poly(n-vinyl pyrrolidone)
Poly(ortho esters)
Polyalkyl-2-cyanoacrylates
Polyanhydrides
Polycyanoacrylates
Polydepsi peptides
Polydihydropyrans
Poly-dl-lactide (PDLLA)
Polyesteramides
Polyesters of oxalic acid
Polyglycolide (PGA)
Polyiminocarbonates
Polylactides (PLA)
Poly-l-lactide (PLLA)
Polyorthoesters
Poly-p-dioxanone (PDO)
Polypeptides
Polyphosphazenes
Polysaccharides
Polyurethanes (PU)
Polyvinyl alcohol (PVA)
Poly-β-hydroxypropionate (PHPA)
Poly-β-hydroxybutyrate (PBA)
Poly-σ-valerolactone
Poly-β-alkanoic acids
Poly-β-malic acid (PMLA)
Poly-ε-caprolactone (PCL)
Pseudo-Poly(Amino Acids)
Starch
Trimethylene carbonate (TMC)
Tyrosine based polymers

TABLE 2
Examples of
Biologically Active Agents Deliverable via the Present Invention
Adenovirus with or without genetic material
Angiogenic agents
Angiotensin Converting Enzyme Inhibitors (ACE inhibitors)
Angiotensin II antagonists
Anti-angiogenic agents
Antiarrhythmics
Anti-bacterial agents
Antibiotics
Erythromycin
Penicillin
Anti-coagulants
Heparin
Anti-growth factors
Anti-inflammatory agents
Dexamethasone
Aspirin
Hydrocortisone
Antioxidants
Anti-platelet agents
Forskolin
Anti-proliferation agents
Anti-rejection agents
Rapamycin
Anti-restenosis agents
Antisense
Anti-thrombogenic agents
Argatroban
Hirudin
GP IIb/IIIa inhibitors
Anti-virus drugs
Arteriogenesis agents
acidic fibroblast growth factor (aFGF)
angiogenin
angiotropin
basic fibroblast growth factor (bFGF)
Bone morphogenic proteins (BMP)
epidermal growth factor (EGF)
fibrin
granulocyte-macrophage colony stimulating factor (GM-CSF)
hepatocyte growth factor (HGF)
HIF-1
insulin growth factor-1 (IGF-1)
interleukin-8 (IL-8)
MAC-1
nicotinamide
platelet-derived endothelial cell growth factor (PD-ECGF)
platelet-derived growth factor (PDGF)
transforming growth factors alpha & beta (TGF-.alpha., TGF-beta.)
tumor necrosis factor alpha (TNF-.alpha.)
vascular endothelial growth factor (VEGF)
vascular permeability factor (VPF)
Bacteria
Beta blocker
Blood clotting factor
Bone morphogenic proteins (BMP)
Calcium channel blockers
Carcinogens
Cells
Bone marrow cells
Blood cells
Stem Cells
Umbilical cord cells
Fat cells
Bone cells
Cartilage cells
Chemotherapeutic agents (e.g. Ceramide, Taxol, Cisplatin)
Cholesterol reducers
Chondroitin
Collagen Inhibitors
Colony stimulating factors
Coumadin
Cytokines prostaglandins
Dentin
Etretinate
Genetic material
Glucosamine
Glycosaminoglycans
GP IIb/IIIa inhibitors
L-703,081
Granulocyte-macrophage colony stimulating factor (GM-CSF)
Growth factor antagonists or inhibitors
Growth factors
Acidic fibroblast growth factor (aFGF)
Autologous Growth Factors
Basic fibroblast growth factor (bFGF)
Bone morphogenic proteins (BMPs)
Bovine Derived Growth Factors
Cartilage Derived Growth Factors (CDF)
Endothelial Cell Growth Factor (ECGF)
Epidermal growth factor (EGF)
Fibroblast Growth Factors (FGF)
Hepatocyte growth factor (HGF)
Insulin-like Growth Factors (e.g. IGF-I)
Nerve growth factor (NGF)
Platelet Derived endothelial cell growth factor (PD-ECGF)
Platelet Derived Growth Factor (PDGF)
Recombinant NGF (rhNGF)
Recombinant Growth Factors
Tissue Derived Cytokines
Tissue necrosis factor (TNF)
Transforming growth factors alpha (TGF-alpha)
Transforming growth factors beta (TGF-beta)
Tumor necrosis factor alpha (TNF-.alpha.)
Vascular Endothelial Growth Factor (VEGF)
Vascular permeability factor (UPF)
Growth hormones
Heparin sulfate proteoglycan
HMC-CoA reductase inhibitors (statins)
Hormones
Erythropoietin
Immoxidal
Immunosuppressant agents
inflammatory mediator
Insulin
Interleukins
Interlukin-8 (IL-8)
Interlukins
Lipid lowering agents
Lipo-proteins
Low-molecular weight heparin
Lymphocites
Lysine
MAC-1
Morphogens
Nitric oxide (NO)
Nucleotides
Peptides
PR39
Proteins
Prostaglandins
Proteoglycans
Perlecan
Radioactive materials
Iodine-125
Iodine-131
Iridium-192
Palladium 103
Radio-pharmaceuticals
Secondary Messengers
Ceramide
Somatomedins
Statins
Steroids
Sulfonyl
Thrombin
Thrombin inhibitor
Thrombolytics
Ticlid
Tyrosine kinase Inhibitors
ST638
AG-17
Vasodilator
Histamine
Forskolin
Nitroglycerin
Vitamins
E
C
Yeast
Adipose cells
Blood cells
Bone marrow
Cells with altered receptors or binding sites
Endothelial Cells
Epithelial cells
Fibroblasts
Genetically altered cells
Glycoproteins
Growth factors
Lipids
Liposomes
Macrophages
Mesenchymal stem cells
Progenitor cells
Reticulocytes
Skeletal muscle cells
Smooth muscle cells
Stem cells
Vesicles

TABLE 3
Examples of Materials Suitable as Particulates
Alginate
Calcium
Calcium Phosphate
Calcium Sulfate
Ceramics
Chitosan
Cyanoacrylate
Collagen
Dacron
Demineralized bone
Elastin
Fibrin
Gelatin
Glass & BioGlass
Gold
Hyaluronic acid
Hydrogels
Hydroxy apatite
Hydroxyethyl methacrylate
Hyaluronic Acid
Liposomes
Mesenchymal cells
Nitinol
Osteoblasts
Oxidized regenerated cellulose
Phosphate glasses
Polyethylene glycol
Polyester
Polysaccharides
Polyvinyl alcohol
Platelets, blood cells
Radiopacifiers
Salts
Silicone
Silk
Steel (e.g. Stainless Steel)
Synthetic polymers
Thrombin
Titanium
Silica
Clay
Metals
Aluminum Oxides
Ceramics
Polymers
Metal Oxides

### CONCLUSIONS

The present invention comprises a regenerative complex engineered to maintain and deliver platelets, either non-activated or activated, via a collagen scaffold for a controlled release of growth factors from the platelets. More particularly, in the present invention, the composition may have a fibrous, reinforced, hydrogel-like, formation containing PRP, and may be transplanted to any lesion in need of tissue regeneration, particularly in cases where amplifying the wound healing response of a living being may be favorable, and accordingly, PRP may be activated to induce release of the platelets' stored growth factors, which is useful for tissue regeneration. Accordingly, this regenerative complex is very useful in highly enhancing the bodies wound healing cascade not only by presenting a concentrated platelet population but by providing a native fibrous collagen network to advance the repair process. Also, the unique collagen configuration in the present invention can be used independently as a hemostat.

1)-16) (canceled)

17) A method of preparing an implantable tissue scaffold comprising the steps of:
a. introducing into a mold a composition comprising at least one blood fraction and a collagen fiber component; and
b. allowing the composition to set to form a fiber reinforced clot having the shape of the mold, and
c. separating the fiber reinforced clot and the mold, thereby forming an implantable tissue scaffold that is suturable,
wherein the at least one blood fraction comprises bone marrow aspirate or the at least one blood fraction has been separated from whole blood and comprises platelet poor plasma or platelet rich plasma, and
wherein the collagen fiber component comprises a non-flocculating distribution of collagen fibers having a length of less than 4 mm.

18) The method of claim 17, wherein the non-flocculating distribution of collagen fibers comprises native insoluble fibrous collagen that has been mechanically milled into a non-flocculating form.

19) The method of claim 17, wherein the collagen fiber component comprises a non-flocculating distribution of native collagen fibers having a length of less than 2 mm.

20) The method of claim 17, wherein the collagen fiber component comprises a non-flocculating distribution of native collagen fibers having a length of between 0.01 mm and 1 mm.

21) The method of claim 17, wherein the at least one blood fraction comprises platelets and plasma at a ratio of from 1:2 to 1:10.

22) The method of claim 17, wherein the composition further comprises a biologically active agent comprising a drug or a growth factor incorporated into the material as an additive.

23) The method of claim 17, wherein the collagen fiber component is cross-linked.

24) The method of claim 17, further comprising the step of:
d. implanting the implantable tissue scaffold into a wound site of a living being.

25) The method of claim 24, further comprising the step of:
e. securing the implantable tissue scaffold to the living being.

26) The method of claim 24, further comprising the step of:
e. securing the implantable tissue scaffold to the living being with a suture.

27) The method of claim 17, wherein the at least one blood fraction is formed by placing said blood sample in said centrifuge device and operating said centrifuge device to fractionate the blood sample into a plurality of blood fractions.

28) The method of claim 17, wherein the at least one blood fraction comprises platelet-rich plasma, and the method further comprising the step of adding an anti-coagulant to the platelet-rich plasma prior to contacting the at least one blood fraction and the collagen fiber component.

29) The method of claim 28, further comprising adding a source of calcium ion to the at least one blood fraction prior to contacting the at least one blood fraction and the collagen fiber component.

30) The method of claim 17, further comprising the step of partially hydrating the collagen fiber component with a non-settable fluid prior to contacting the at least one blood fraction and the collagen fiber component.

31) An implantable tissue scaffold formed from the method of claim 17.

32) The method of claim 19, wherein the non-flocculating distribution of collagen fibers comprises native insoluble fibrous collagen that has been mechanically milled into a non-flocculating form.

33) An implantable tissue scaffold formed from the method of claim 32.

34) The method of claim 20, wherein the non-flocculating distribution of collagen fibers comprises native insoluble fibrous collagen that has been mechanically milled into a non-flocculating form.

35) An implantable tissue scaffold formed from the method of claim 34.

36) A method of treating a wound in a living being comprising the steps of:
a. providing an implantable tissue scaffold formed from the method of claim 17, and
b. suturing the implantable tissue scaffold to implanting a wound site of a living being.
